SEQUENCE LISTING (2 sequences)

SEQ ID NO: 1 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

(19) United States
(12) Patent Application Publication
(10) Pub. No.: US 2019/0309054 A1
(43) Pub. Date: Oct. 10, 2019

(54) NON-POLYAMINATED LCN2 AS A BIOMARKER FOR DIAGNOSIS AND TREATMENT OF CARDIOMETABOLIC DISEASES

(71) Applicant: THE UNIVERSITY OF HONG KONG, Hong Kong (CN)

(72) Inventors: Yu WANG, Hong Kong (CN); Aimin XU, Hong Kong (CN); Kangmin YANG, Hong Kong (CN); Wing Chung MAN, Hong Kong (CN)

(73) Assignee: THE UNIVERSITY OF HONG KONG, Hong Kong (CN)

(21) Appl. No.: 16/345,432

(22) PCT Filed: Oct. 28, 2016
(86) PCT No.: PCT/CN2016/103792

§ 371 Date: Apr. 26, 2019

(51) Int. Cl. C07K 16/18; G01N 33/68; G01N 33/493

(52) U.S. Cl. CPC C07K 16/18; G01N 33/6893; G01N 2800/32; G01N 2800/042; G01N 33/493

(57) ABSTRACT

The invention provides the use of non-polyaminated lipocalin-2 and/or polyaminated lipocalin-2 as biomarkers for cardiometabolic disease as well as antibodies, assays and devices related to these biomarkers.

### BACKGROUND OF THE INVENTION

Lipocalin-2 (Lcn2), also known as neutrophil gelatinase associated lipocalin, neu-related lipocalin, uterocalin, siderocalin or 24p3, is a protein of 198 amino acids belonging to the lipocalin family of proteins that function as transporters of lipophilic substances^{1, 2}. Lcn2 possesses unique bacteriostatic properties by sequestering enterobactin³, and is implicated in cardiometabolic abnormalities associated with obesity, such as hypertension, diabetes, renal injury and heart failure⁴⁻¹². Circulating Lcn2 levels are significantly augmented in obese human subjects and positively correlated with anthropometric metabolic variables including insulin resistance, hyperlipidemia, hyperglycemia and inflammation⁶. Mice without Lcn2 are protected from dietary or genetic obesity-induced endothelial dysfunction, hypertension, insulin resistance, and elevation of circulating lipid and glucose levels^{4, 5, 12-14}.

Lcn2 is post-translationally modified by polyamination, which promotes the clearance of this protein from circulation. After replacing the cysteine 87 residue by alanine in human Lcn2, the amount of polyamines attached to the mutant (C87A) is significantly reduced⁵. C87A exhibits a much longer plasma half-life than wild type human Lcn2. Injection of C87A leads to the accumulation of Lcn2 protein in blood vessel wall, which causes endothelial dysfunction and vascular inflammation in mice fed with standard chow⁵.

### BRIEF SUMMARY OF THE INVENTION

The instant invention provides Lcn2, particularly, non-polyaminated Lcn2 (npLcn2) as a biomarker for identification or risk assessment of cardiometabolic diseases. Accordingly, assays, for example, immunoassays, for detection and quantification of polyaminated Lcn2 (pLcn2) and/or npLcn2 in different human tissues and biofluids are provided.

An embodiment of the invention provides an assay for determining the level of pLcn2 and/or npLcn2 in a body fluid of a subject. The level of pLcn2 and/or npLcn2 in a body fluid of a subject can be used for identifying a subject as having, not having, having a high risk of, or not having a high risk of developing a cardiometabolic disease.

The invention also provides kits and reagents to conduct assays to quantify npLcn2 and/or pLcn2.

In another embodiment, the invention provides a method of treating and/or managing and/or preventing a cardiometabolic disease in a subject by administering to the subject a pharmaceutically effective amount of an antibody against npLcn2 and/or an antibody against pLcn2.

### BRIEF DESCRIPTION OF THE DRAWINGS

FIG. 1. Comparison of Lcn2 concentrations stratified by the number of the components of metabolic syndrome. Both pLcn2 (left) and npLcn2 (right) levels were measured and compared for the plasma (top) and pericardial fluid (bottom) samples of Danish subjects. *, p<0.05 versus the group with zero component of metabolic syndrome (n=8-15).

FIG. 2. Comparison of samples stratified by the pattern of npLcn2 distribution in pericardium tissue biopsies. A, Immunohistochemical staining was performed for tissue sections using polyclonal antibodies against npLcn2. B, Based on the number of positively stained cells and the distribution pattern, samples were separated into three groups for comparison of their Lcn2, total cholesterol (TC) and plasma creatinine levels. *, p<0.05 (n=9-12). Arrows indicate the positive staining of npLcn2. A=adipocytes, M=mesothelial cells, L=leukocytes. Magnification, 200×.

FIG. 3. Polyclonal antibodies against wild type human Lcn2 or C87A selectively recognize different species of Lcn2. Equal amount of purified wild type human Lcn2 and C87A mutant were mixed in one test tube and then incubated with anti-human Lcn2 or anti-C87A mutant antibody for six hours at 4° C. Subsequently, 70 μl of protein A sepharose bead slurry was added for immunoprecipitation. The precipitated proteins were separated in 15% SDS-PAGE and detected with anti-pLcn2 antibody.

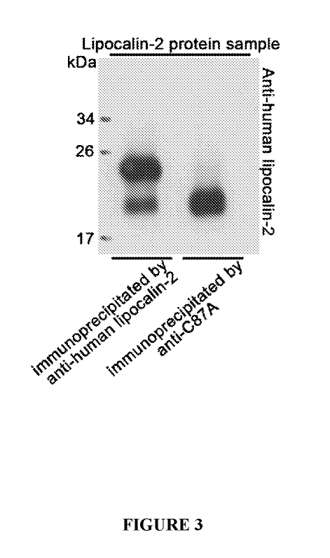

FIG. 4. Detection of polyaminated and non-polyaminated Lcn2 in human urine samples. A, The concentrated urine samples were separated by SDS-PAGE (15 μl/lane) and then subjected to Western blotting detection by polyclonal antibodies against pLcn2 (anti-pLcn2) or npLcn2 (anti-npLcn2). B, Immunoprecipitation (ip) was performed in two of the nine urine samples using anti-pLcn2 and anti-npLcn2, respectively. Polyamines attached to the precipitate Lcn2 protein were detected using antibodies recognizing spermidine.

FIG. 5. The frequency distribution of serum (top) and urine (bottom) lipocalin-2 concentrations for samples of the Hong Kong healthy volunteer cohort. The 95^{th} and 75^{th} percentile values are indicated for both pLcn2 (left) and npLcn2 (right) levels.

FIG. 6. Representative images of tissue sections stained by polyclonal antibodies against pLcn2 (left) or npLcn2 (right). The parietal pericardium tissue biopsies were collected from Danish subjects during elective coronary artery bypass grafting or cardiac valve replacement surgery and subsequently processed for immunohistological analyses. Arrows indicate the different types of cells with pLcn2 or npLcn2 positive staining (brown color). A=Adipocytes, M=Mesothelial cells, L=Leukocytes.

FIG. 7. Comparison of cardiometabolic parameters among the three groups of samples. Based on the number of positively stained cells and the distribution pattern of npLcn2 (FIG. 2A), samples from Danish subject cohort were separated into three groups for comparison of their high-density lipoprotein cholesterol (HDL), low-density lipoprotein cholesterol LDL, hemoglobin A1c (HbA1c) and plasma adiponectin levels. (n=9-12)

### BRIEF DESCRIPTION OF THE SEQUENCES

SEQ ID NO: 1: Sequence of human full-length Lcn2.

SEQ ID NO: 2: Sequence of human Lcn2 lacking first 20 amino acids that form a single peptide. The cysteine 87 residue referred elsewhere in this disclosure is with respect to the mature circulating Lcn2 protein having the sequence of SEQ ID NO: 2, which is the mature human Lcn2 protein lacking the signal peptide.

### DETAILED DISCLOSURE OF THE INVENTION

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of ingredients where the terms “about” or “approximately” are used, these compositions contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0,4, 0.5, 0.6, 0.7, 0.8, 0.9, and all intermediate ranges encompassed within 0.1-4.0, such as 0.2-0.5, 0.2-0.8, 0.7-1.0, etc.

When ranges are used herein, such as for dose ranges, combinations and subcombinations of ranges (e.g., subranges within the disclosed range), specific embodiments therein are intended to be explicitly included.

“Treatment” or “treating” and grammatical variants of these terms refer to an approach for obtaining beneficial or desired results including but not limited to therapeutic benefit. A therapeutic benefit is achieved with the eradication or amelioration of one or more of the pathological symptoms associated with a cardiometabolic disease, such that an improvement is observed in the patient, notwithstanding that the patient may still be afflicted with the cardiometabolic disorder.

“Management” or “managing” and grammatical variants of these terms refer to an approach for stopping the worsening of symptoms associated with a cardiometabolic disease or the progression of a cardiometabolic disease.

“Prevention” or “preventing” and grammatical variants of these terms refer to an approach for preventing the occurrence of a cardiometabolic disease, for example, in a subject identified as having high risk of developing a cardiometabolic disease.

The term “cardiometabolic disease” refers to a disease of cardiac system caused by a clustering of interrelated risk factors that promote the development of atherosclerotic vascular disease and/or type 2 diabetes mellitus. Certain aspects of a cardiometabolic disease include hypertension, diabetes, renal injury, heart failure, insulin resistance, hyperlipidemia, hyperglycemia, inflammation, vascular inflammation, endothelial dysfunction, increased cholesterol, increased HbA1c, reduced HDL, reduced adiponectin, atherosclerosis, and diseases of cardio-renal axis. A cardiometabolic disease as used herein does not require the presence of all of the aspects listed above and certain aspects may be absent and additional aspects not listed herein may be present.

Cardiorenal syndromes are disorders of the heart and kidneys whereby acute or long-term dysfunction in one organ may induce acute or long-term dysfunction of the other. Cardiorenal syndromes is characterized by the concomitant decreased kidney function, therapy-resistant heart failure with congestion and worsening kidney function during heart failure therapy.

The phrase “a subject having high risk of developing a cardiometabolic disease” indicates that the subject is more likely than not to develop the cardiometabolic disease. A subject having high risk of developing a cardiometabolic disease is more likely to develop a cardiovascular disease within about one year to two years, particularly, within about six months to a year, more particularly, within about a year from being identified as having high risk of developing a cardiometabolic disease.

The phrase “a subject having low risk of developing a cardiometabolic disease” indicates that the subject is more likely than not to be free from the cardiometabolic disease. A subject having low risk of developing a cardiometabolic disease is more likely to be free from a cardiovascular disease for at least about one year to two years, particularly, at least about six months to a year, more particularly, at least about a year from being identified as having low risk of developing a cardiometabolic disease.

“Subject” refers to an animal, such as a mammal. The methods described herein can be useful in both humans and non-human mammals.

The term Lipocalin-2 (Lcn2) as used herein refers to Lcn2 protein that represents a sum of polyaminated or non-polyaminated Lcn2. The term “polyaminated Lcn2 (pLcn2)” refers to a portion of Lcn2 protein that is polyaminated; whereas, the term “non-polyaminated Lcn2 (npLcn2)” refers to a portion of Lcn2 protein that is not polyaminated.

Lcn2 is characterized by a highly diversified expression pattern and structure-functional relationship. The mRNA and protein of Lcn2 are either constitutively expressed or induced during injury, differentiation, maturation or transformation of different types of cells, including adipocytes^{30, 31}, neutrophils^{2, 27}, macrophage^{32, 33}, normal and malignant epithelial cells³⁴⁻³⁷. The expression of Lcn2 is subjected to extensive regulation. For instance, Lcn2 protein is synthetized from the gene transcript in myelocytes and metamyelocytes during the maturation, and then stored in specific granules of neutrophils³⁸⁻⁴⁰. Thus, a high level of Lcn2 mRNA expression is detected in bone marrow but not in peripheral leukocytes, including the tissue-infiltrating neutrophils^{36, 41}. In adipocytes and hepatocytes, Lcn2 is constitutively expressed at both gene and protein levels, which represent a major source of this molecule in circulation under physiological conditions^{42, 43}. In epithelial cells, Lcn2 is induced predominantly by involution, injury or dysplastic transformation, and modulates the phenotype of the epithelial lineage in growth and diseases^{44, 45}.

At the tissue level, Lcn2 levels are influenced by development, ageing, infection and inflammation status^{41, 44-47}. In physiological conditions, Lcn2 is undetectable or present at very low levels in tissues such as heart and kidney. In response to injury, infection or other pathological conditions, increased Lcn2 expression is found in tissues including kidney, heart, liver, colon and breast⁴⁸. Significantly augmented Lcn2 levels are positively associated with systemic diseases absence of overt bacterial infection, such as insulin resistance, hypertension, type 2 diabetes mellitus and other obesity-related pathologies^{6, 14, 49, 50}.

In circulation, Lcn2 exists as a 25 kDa monomer, a 46 kDa homodimer, or a 130 kDa heterodimer with matrix metalloproteinase-9⁵¹. The clearance of the monomeric form is more rapid than the dimeric form of Lcn2⁵². In urine, Lcn2 is also presented as multiple molecular forms^{7, 43, 44}. Under physiological conditions, circulating Lcn2 is filtered by glomeruli and the protein captured in the proximal tubular epithelial cells of kidney^{45, 46}. Thus, when injecting into the circulation, Lcn2 is enriched in the proximal tubule but does not appear in the urine in large quantities⁵³. This is in line with the present study that serum and urinary Lcn2 levels are not significantly correlated with each other in healthy human subjects.

Human and murine Lcn2 are modified by polyamination⁵. The amount of polyamines attached to Lcn2 determines its circulating half-life and biological activities. Using an antibody against wild type human Lcn2 that recognizes pLcn2 and an antibody against C87A mutant Lcn2 that specifically binds to npLcn2, the present invention demonstrates that the endogenous Lcn2 exists as both polyaminated and non-polyaminated forms in human samples including serum, plasma, urine and pericardial fluids. The distribution and concentration of the two forms of Lcn2 are closely associated with the metabolic and inflammatory, as well as the cardio-renal functional status of healthy human subjects and patients with cardiac abnormalities. Compared to pLcn2, the serum and urinary npLcn2 levels are more sensitively correlated with BMI, HR, and TG levels in healthy volunteers. In subjects undergoing cardiothoracic surgery, the circulating concentrations of npLcn2 are significantly elevated and correlated with risk factors such as CRP and FGF21. Moreover, significantly increased local expression of npLcn2 in pericardium of Group III subjects is highly correlated with augmented LDL, TC, HbA1c and creatinine, but reduced HDL and adiponectin levels.

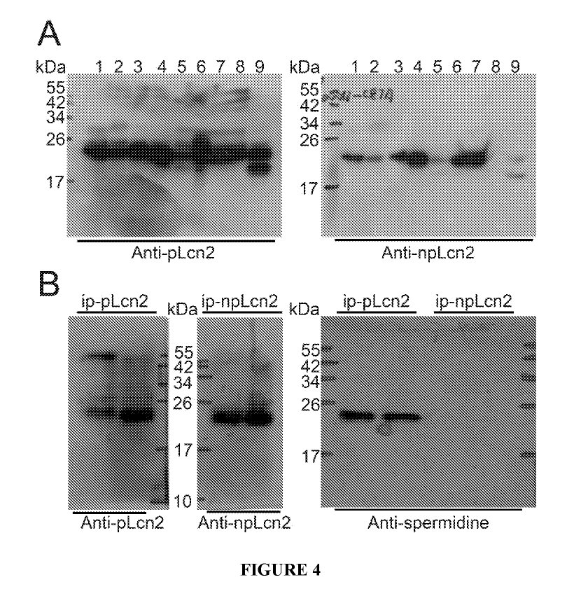

Only few studies reported the reference ranges of circulating and urinary Lcn2 levels in normal subjects⁵⁴⁻⁵⁶. The invention describes the reference urinary Lcn2 levels in a healthy cohort of subjects and a non-hospitalized Chinese population. Subjects with metabolic syndrome have elevated pLcn2 and npLcn2 levels in urine. Among the subjects with urinary pLcn2 levels higher than 106.0 ng/ml, the prevalence of metabolic syndrome is 20 percent, and among those with higher than 395.1 ng/ml, the prevalence increases to 33.3 percent. Similarly, subjects having urinary npLcn2 levels higher than 9.1 ng/ml or 26.0 ng/ml, the prevalence of metabolic syndrome are 24 and 60 percent, respectively (FIG. 4).

In response to ischemic or nephrotoxic injury, various protein markers appear in urine as a result of impaired tubular reabsorption or catabolism of filtered molecules and abnormal release of components from tubular cells^{57, 58}. The rapid rise of urinary Lcn2 is an early biomarker for acute renal failure, which occurs in up to 40% of adults after cardiac surgery and complicates up to 10% cardiac surgical procedures in infants and children with congenital heart disease^{53, 54}. Urinary Lcn2 in subjects without kidney injury decreases rapidly after cardiac surgery⁵⁹. Measurement of urinary Lcn2 not only provides early diagnosis of acute kidney injury, but also predicts clinical outcomes, such as dialysis requirement and mortality⁶⁰. By contrast, serum levels of Lcn2 are inferior to those in urine for the identification of acute kidney injury.

In adult subjects, the comorbid conditions such as advanced age and related diseases (e.g. atherosclerosis or diabetes) prevent a clear-cut between subjects who have or have not develop acute kidney injury⁶¹. These uncertainties are largely attributed to the unknown origin and structure of the multiple forms of urinary Lcn2. Lcn2 in urine samples of subjects with acute kidney injury represents a collection of different pools of the protein, including those freely filtered into the tubular space, released from injured tubular cells, as well as locally expressed and excreted⁶¹. After renal injury, Lcn2 mRNA is predominantly expressed in the loop of Henle and collecting ducts^{57, 58}. This locally synthesized Lcn2 is unlikely to be introduced back into the circulation but rather to be excreted into the urine⁶². Paradoxically, Lcn2 protein in the postischemic kidney is mainly localized at the damaged proximal tubule⁶³. Cultured glomeruli and glomerular mesangial cells secret Lcn2 in response to macrophage stimulation or cytokine treatment³².

Plasma Lcn2 concentration increases progressively with the reduction of glomerular filtration rate (GFR), due to the impaired removal of Lcn2 from circulation⁵⁴. When GFR drops, urinary Lcn2 may represent mainly those that are locally expressed. A third source of Lcn2 may be activated neutrophils/macrophages or inflamed vasculature. Theoretically, the different sources of Lcn2 should correlate with the stages of disease progression. For instance, in subjects with acute decompensated heart failure, urinary Lcn2 levels reflect renal distal tubular injury with impaired natriuresis and diuresis, whereas systemic Lcn2 levels demonstrate a stronger association with glomerular filtration function. Both systemic and urinary Lcn2 predict worsening renal function. However, understanding the contribution of individual pools of Lcn2 synthesized in response to renal injury is important for both risk assessment and disease management. Thus, the measurement of both pLcn2 and npLcn2 provides more insights and accurate information for clinical evaluations.

The reasons for the development of acute kidney injury after cardiac surgery or the association of chronic kidney disease with heart failure are not completely understood; however, these are probably related to common risk factors for the diseases of cardio-renal axis. Lcn2 represents an important link between renal and cardiovascular system dysfunctions. Urinary and plasma Lcn2 levels are highly increased in subjects with cardio-renal syndrome types 1 and 2⁶⁴⁻⁶⁷. Lcn2 is a stronger predictor for mortality than GRF and cystatin C in subjects with heart failure. Moreover, the prognostic effect of Lcn2 on long-term mortality is limited to those with normal GRF on admission. Raised Lcn2 levels are observed in subjects with normal serum creatinine. Thus, the high Lcn2 levels do not merely reflect impaired renal functions, but may be related to the cardiac abnormality per se. In fact, urinary and plasma Lcn2 levels are positively associated with increased N terminal-pro brain natriuretic peptide (NT-proBNP), NYHA class and LVIDd (left ventricular internal end-diastolic dimension) in subjects with chronic heart failure^{37, 39}. Plasma Lcn2 levels correlate with heart failure severity and predict major adverse cardiovascular events in critically ill subjects. Moreover, a recent study suggests that Lcn2 levels are positively and significantly associated with coronary artery disease severity in subjects without heart failure and renal dysfunction.

The present invention demonstrates that in healthy subjects, plasma npLcn2 levels correlate with HR, independent of age, gender, smoking and BMI. In cardiothoracic surgery subjects, plasma and pericardial fluid npLcn2 are significant elevated, much higher than pLcn2 levels in both male and female subjects. Moreover, there is positive association between serum creatinine levels and Lcn2 contents in pericardial fluids. A significant amount of npLcn2 is present in pericardial fluid collected from Danish subjects and positively correlated with those in plasma samples. Histological studies confirmed the presence of npLcn2 protein in the mesothelial cell layer and the underlying connective tissues (adipocytes) of at least one third subject biopsies. Among two third of the tissue sections contained positively stained blood cells (leukocytes) mainly in venules or lymphatic vessels but not arterioles. Although pLcn2 and npLcn2 were both detectable in the leukocytes, their patterns of distribution differed significantly as judged from the staining results of adjacent tissue sections. Considering that the mesothelial cells possess the capacity of differentiation into adipocytes^{68, 69}, npLcn2 expression in these two types of cells may play a role in modulating the pericardial fat content and function, as well as the inflammatory status.

Pericardium is a fibrous-serosal cavity surrounding the heart that contains a small amount of fluid⁷⁰. In addition to anatomic isolation and lubricating the moving surfaces of the heart, normal pericardium prevents cardiac hypertrophy in pressure overload conditions and preserves the negative endothoracic pressure for atria blood filling⁷¹. The contents of human pericardial fluid have not been defined, but considered to be produced by plasma ultrafiltration via the epicardial capillaries. Various blood cells are present in pericardial fluid⁷². A monolayer of flattened, squamous-like mesothelial cells line the inner surface of pericardial cavity and play a role in absorbing the pericardial fluid for drainage through the lymphatic capillary bed. Under pathological conditions, mesothelial cells secret pro-inflammatory and pro-fibrotic mediators, differentiate and migrate into the surrounding tissues to promote fibrogenesis.

The invention describes that npLcn2 expression is induced locally in the mesothelial cells and pericardial adipocytes. Leukocytes such as neutrophils or macrophages act as scavengers to clear npLcn2 protein from the pericardium. During this process, npLcn2 is polyaminated inside the leukocytes and stored in the form of pLcn2. Excessive production and accumulation of npLcn2 may facilitate the development of cardiac abnormalities. Collectively, locally produced npLcn2 not only determines systemic Lcn2 levels but also plays a pathogenic role in the development of cardiometabolic diseases, for example, heart diseases. Thus, measurement of different forms of Lcn2, especially npLcn2, is clinically important for early detection, risk stratification and outcome prediction of subjects with cardio-renal abnormalities. As such, npLcn2 is presented as a novel and sensitive biomarker for cardio-renal function assessment and for diagnosing and treating and/or managing and/or preventing a cardiometabolic disease.

Accordingly, an embodiment of the invention provides a method for treating and/or managing and/or preventing a cardiometabolic disease in a subject, the method comprising the steps of:

(a) determining the level of npLcn2 in:
i) a test sample obtained from the subject, and
ii) optionally a control sample;

(b) optionally, obtaining one or more reference values corresponding to the level of npLcn2; and

(c) identifying the subject as:
i) having or having high risk of developing a cardiometabolic disease based on the level of npLcn2 in the test sample and optionally, administering to the subject a therapy designed to treat and/or manage and/or prevent the cardiometabolic disease, or
ii) identifying the subject as not having or having low risk of developing the cardiometabolic disease based on the level of npLcn2 in the test sample and withholding from the subject the therapy designed to treat and/or manage and/or prevent the cardiometabolic disease

Various techniques are well known to a person of ordinary skill in the art to determine the level of npLcn2 in a sample. Non-limiting examples of such techniques include antibody based assay or protein mass-spectrometry. Certain techniques of spectrometric analysis of proteins are described in the Harvey (2005) reference, which is herein incorporated by reference in its entirety.

Non-limiting examples of the antibody based assays include Western blotting analysis, enzyme immunoassay (EIA), enzyme linked immunosorbent assay (ELISA), radioimmune assay (RIA), immunohistological analysis, and antigen-antibody precipitation assay. Additional examples of antibody-based assays are for determining the level of npLcn2 in a sample are well known to a person of ordinary skill in the art and such embodiments are within the purview of the invention.

The reference values corresponding to the level npLcn2 in a sample may indicate the level of npLcn2 associated with no risk or low risk of developing a cardiometabolic disease or the presence or high risk of developing a cardiometabolic disease. As such, a reference value corresponding to a level of npLcn2 in a sample may indicate the absence, presence, high risk of developing or low risk of developing a cardiometabolic disease.

In one embodiment, the step of identifying the subject as having, not having, having low risk of developing or having high risk of developing a cardiometabolic disease depends on the level of npLcn2 in the test sample compared to a control sample. For example, if the level of npLcn2 in the test sample is significantly higher than the level of npLcn2 in the control sample obtained from a healthy individual, the subject is identified as having a cardiometabolic disease or having a high risk of developing of a cardiometabolic disease. Particularly, a subject is identified as having a cardiometabolic disease or having high risk of developing a cardiometabolic disease if the level of npLcn2 is higher in a blood, serum, urine, plasma, or pericardial fluid sample of a subject compared to the level in a corresponding control sample obtained from a healthy individual.

An embodiment of the invention provides an antibody or an antigen binding fragment of the antibody that specifically binds to npLcn2. In an embodiment, the antibody is labeled, for example, with a detectable label such as an enzyme label, a radioisotope, a fluorescent label, or a bioluminescent label. The labels are typically used for detection and visualization of antigen-antibody complex. Non-limiting examples of the enzyme labels are horse-radish peroxidase label, alkaline phosphatase, β-galactosidase, luciferase, acetylcholine esterase, and glucose oxidase. Additional examples of enzymes appropriate for labeling antibodies for detection and visualization of antigen-antibody complex are well known to a person of ordinary skill in the art and such embodiments are within the purview of the current invention. Non-limiting examples of radioisotope labels are 125I, ³⁵S, ¹⁴C, ³²P and ³H. Additional examples of radiolabels appropriate for labeling antibodies for detection and visualization of antigen-antibody complex are well known to a person of ordinary skill in the art and such embodiments are within the purview of the current invention. Non-limiting examples of fluorescent labels are umbelliferone, fluorescein, rhodamine, dichlorotriazinylamine fluorescein, dansyl chloride, fluorescein isothiocyante (FITC), phycoerythrin (PE), Cy5-phycoerythrin (Cy5-PE), Cy7-phycoerythrin (Cy7-PE), allophycocyanin (APC), Cy7-allophycocyanin (Cy7-APC), texas red (TR) and cascade blue. Additional examples of fluorescent labels appropriate for labeling antibodies for detection and visualization of antigen-antibody complex are well known to a person of ordinary skill in the art and such embodiments are within the purview of the current invention. Non-limiting examples of bioluminescent labels are photoprotein aequorin, adenosine triphosphate, nicotinamide adenine dinucleotide and D-luciferin. Additional examples of bioluminescent labels appropriate for labeling antibodies for detection and visualization of antigen-antibody complex are well known to a person of ordinary skill in the art and such embodiments are within the purview of the current invention.

Examples of various protocols of selecting antigens for raising antibodies, immunizing animals to produce antibodies, generating monoclonal antibodies, growing hybridoma producing the monoclonal antibodies, purifying and storing the antibodies, labeling the antibodies, conducting the antibody-based assays, engineering hybrid antibodies, and staining cells with labeled antibodies are described in Greenfield, E., (2014), Antibodies: A Laboratory Manual, Second Edition, Cold Spring Harbor, N.Y.: Cold Spring Harbor Laboratory Press. The Greenfield (2014) reference is herein incorporated in its entirety. Additional protocols of performing these techniques are well known to a person of ordinary skill in the art and such embodiments can also be used in practicing the current invention.

The term “antibody” as used herein refers to immunoglobulin molecules and immunologically active portions of immunoglobulin molecules, i.e., molecules that contain an antigen binding site which specifically binds an antigen. “Specific binding” or “specificity” as used herein refers to the ability of an antibody to exclusively bind to an epitope presented on an antigen or peptide while having relatively little non-specific affinity with other proteins or peptides. Specificity can be relatively determined by binding or competitive binding assays. Specificity can be mathematically calculated by, e.g., an about 10:1, about 20:1, about 50:1, about 100:1, 10.000:1 or greater ratio of affinity/avidity in binding to the specific antigen or peptide versus nonspecific binding to other irrelevant molecules. An antibody specifically binding to an antigen has the equilibrium dissociation constant (K_{D}) of lower than about 10⁻⁶ M, lower than about 10⁻⁹ M, or lower than about 10⁻¹² M for the binding between the antibody and the corresponding antigen.

On the other hand, “non-specific binding” refers to the binding that is not based on specific interactions between an antibody and its corresponding antigen. Non-specific binding may result from non-specific interactions, such as, Van Der Waals forces. K_{D} for the binding between the antibody and a non-specific antigen is typically higher than about 10⁻⁶ M, higher than about 10⁻⁴ M or higher than about 10⁻² M.

The invention provides polyclonal and monoclonal antibodies that bind npLcn2. The term “monoclonal antibody” as used herein refers to antibodies made by identical immune cells that are clones of a unique parent cell. Therefore, the amino acid sequences of various molecules of monoclonal antibodies are identical. The term “polyclonal antibody” as used herein refers to antibodies that are secreted by different B cell lineages. Therefore, polyclonal antibodies are a collection of immunoglobulin molecules that bind to a specific antigen, each identifying a different epitope. Some polyclonal antibodies may bind to the same epitope; however, have different amino acid sequences because they are secreted by different B cell lineages. In one embodiment, the antibody or the antigen binding fragment of the antibody is specific to human npLcn2. Human npLcn2 protein has the sequence of SEQ ID NO: 1; whereas, human pLcn2 is polyaminated on possibly more than one residue.

Further embodiments of the invention provide recombinant antibodies or hybrid antibodies. Recombinant or hybrid antibodies typically comprise both human and non-human portions and which can be made using standard recombinant DNA techniques well known to a person of ordinary skill in the art. Non-limiting examples of recombinant or hybrid antibodies include chimeric antibodies, humanized monoclonal antibodies, a single chain antibody, a single chain fragment variable (scFv) antibody, or a fragment antigen-binding (Fab fragment).

A further embodiment of the invention also provides a kit comprising an antibody or an antigen binding fragment of the antibody that specifically binds to npLcn2. The kit can contain the antibody along with additional reagents required for processing of a sample for the immunoassay, reagents for conducting the immunoassay and instructional materials and manuals for performing the immunoassay. Reagents for treating the samples can include reagents for extraction of proteins, degradation of DNA, or removal of other impurities.

An aspect of the invention provides a point-of-care (POC) diagnostic device for assaying npLcn2, which can be used to identifying the subject as having, not having, having low risk of developing or having high risk of developing a cardiometabolic disease.

To practice the methods described herein control samples can be obtained from one or more of the following:

a) an individual belonging to the same species as the subject and not having a cardiometabolic,

b) an individual belonging to the same species as the subject and known to have a low risk or no risk of developing a cardiometabolic, or

c) the subject prior to getting a cardiometabolic.

Additional examples of control samples are known to a person of ordinary skill in the art and such embodiments are within the purview of the current invention.

In certain embodiments, the control sample and the test sample are obtained from the same type of an organ or tissue. Non-limiting examples of the organ or tissue which can be used as samples are brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, or blood. Additional examples of organs and tissues are well known to a person of ordinary skill in the art and such embodiments are within the purview of the invention.

In certain other embodiments, the control sample and the test sample are obtained from the same type of a body fluid. Non-limiting examples of the body fluids which can be used as samples include aqueous humor, vitreous humor, bile, blood, cerebrospinal fluid, chyle, endolymph, perilymph, female ejaculate, lymph, mucus (including nasal drainage and phlegm), pericardial fluid, peritoneal fluid, pleural fluid, pus, rheum, saliva, sputum, synovial fluid, vaginal secretion, semen, blood, serum or plasma. Additional examples of body fluids are well known to a person of ordinary skill in the art and such embodiments are within the purview of the invention.

In one embodiment, once a subject is identified as having or having high risk of developing a cardiometabolic disease based on the methods described herein, a therapy is administered to the subject for treating and/or managing and/or preventing the cardiometabolic disease. In a particular embodiment, the treatment comprises administering an antibody or antibody fragment that specifically binds to Lcn2, particularly, an antibody that specifically binds to npLcn2 or pLcn2. In another embodiment, the therapy is dialysis or to administering an inhibitor such as chemical compounds to interfere npLcn2 expression or activities.

A further embodiment of the invention provides an assay, the assay comprising determining the level of pLcn2 and/or npLcn2 in:
i) a test sample obtained from the subject, and
ii) optionally a control sample.

A particular embodiment of the invention provides an assay for determining whether the level of pLcn2 and/or npLcn2 in a test sample obtained from a subject is above or below a threshold level. In certain embodiments, the sample is a urine sample and the threshold level of pLcn2 is about 100-120, particularly, 110 ng/ml or about 350-450 ng/ml, particularly, 400 ng/ml, the prevalence increases to 33.3 percent. In certain other embodiments, the sample is a urine sample and the threshold level of npLcn2 is about 9-11 ng/ml, particularly, 9.1 ng/ml or 25-30 ng/ml, particularly, 26 ng/ml.

A further embodiment of the invention also provides devices that indicate whether the level of pLcn2 and/or npLcn2 in a test sample obtained from a subject is above or below a threshold level, for example, by providing a signal. For example, the device provides a first signal if the level of pLcn2 and/or npLcn2 in a test sample obtained from a subject is above a threshold level and the device can provide a second signal if the level of pLcn2 and/or npLcn2 in the test sample obtained from the subject is below the threshold level. In another embodiment, the device provides a signal only if the level of pLcn2 and/or npLcn2 in a test sample obtained from a subject is above a threshold level. In a further embodiment, the device provides a signal only if the level of pLcn2 and/or npLcn2 in the test sample obtained from the subject is below a threshold level.

Based on known techniques of detecting the level of pLcn2 and/or npLcn2 in a sample, different devices can be designed by a skilled artisan that provide a signal indicating the level of pLcn2 and/or npLcn2 in the test sample obtained from the subject is above or below a threshold level and such embodiments are within the purview of the invention.

### Materials and Methods

Human Participants

One hundred volunteers including 59 male and 41 female were recruited from Hong Kong. Informed written consent was obtained from all subjects prior to their participation. Criteria of exclusion were pregnancy or lactation, alcohol intake within the past 24-hours, long-term drug treatments or medications taken within one-week prior to the study, any known diagnoses of hypertension, diabetes, dyslipidemia, anemia, coronary heart disease, chronic obstructive pulmonary disease, asthma, hepatitis, primary hyperaldosteronism, renal dysfunction and eczema. The anthropometric parameters including age, body mass index (BMI), waist circumference (WC), heart rate (HR), systolic (SBP) and diastolic (DBP) blood pressures were assessed by following standardized procedures. Recordings are summarized in Table 1 for all participants, including 64 lean (BMI, <25 kg/m²), 35 overweight (BMI, 25-29.9 kg/m²), and one obese (BMI, ≥30 kg/m²) individuals. Thirty-seven subjects who underwent elective coronary artery bypass grafting or cardiac valve replacement surgery were recruited from Southern Denmark. The demographic and clinical characteristics of all study subjects are included in Table 1.

TABLE 1
Characteristics of the study subjects.

| | Hong Kong community | Danish |
|---|---|---|
| | cohort | subjects |
| Parameters | (mean ± SD) | (mean ± SD) |
| Age, years | 48 ± 7 | 69 ± 9 |
| BMI, kg/m² | 23.8 ± 2.7 | 28.7 ± 5.5 |
| WC, cm | 82 ± 7 | — |
| HR | 65 ± 8 | — |
| SBP, mmHg | 117 ± 14 | 135 ± 21 |
| DBP, mmHg | 74 ± 11 | 73 ± 13 |
| FPG, mmol/L | 5.8 ± 0.7 | — |
| TG, mmol/L | 1.7 ± 0.8 | 1.4 ± 0.6 |
| TC, mmol/L | 5.4 ± 0.9 | 3.8 ± 0.9 |
| HDL, mmol/L | 1.6 ± 0.2 | 1.1 ± 0.4 |
| LDL, mmol/L | 3.5 ± 0.9 | 2.1 ± 0.9 |
| Hypertension | — | 70% |
| Dyslipidemia | — | 81% |
| Type 2 diabetes | — | 49% |

Laboratory Analysis

After overnight (10-12 hours) fasting, serum, plasma and urine samples were collected at around 8:00 to 10:00 am for Hong Kong participants. Plasma samples were collected from Danish participants the day before surgery. Pericardial fluid and a biopsy from parietal pericardium of the subjects were collected during the elective coronary artery bypass grafting or cardiac valve replacement surgeries. Tissues biopsies were fixed in neutral-buffered formalin for 48 hours. All biofluid samples were stored at −80° C. until analysis. Fasting plasma glucose (FPG) was analyzed using Accu-Chek Advantage II Glucometer (Roche Diagnostics, Mannheim, Germany). Triglycerides (TG), total cholesterol (TC), high density lipoprotein cholesterol (HDL) and low density lipoprotein cholesterol (LDL) levels in serum samples were analyzed using respective LiquiColor test kits from Stanbio Laboratory, Boerne, Tex., USA. Circulating lipid levels in plasma collected from the Danish subjects were measured at the Odense University Hospital¹⁶.

Immunoassay

Serum or plasma concentrations of adiponectin, Lcn2, C-reactive protein (CRP), fibroblast growth factor 21 (FGF21) and adipocyte fatty acid-binding protein (A-FABP) were performed using in-house ELISA kits (see world-wide website: pharma.hku.hk/sweb/antibody/ELISA.php) as previously described¹⁷⁻²⁶. Leptin was measured using an ELISA from Diagnostic Systems Laboratories, Webster, Tex., USA. Aldosterone levels were determined in serum and urine samples using the DetectX Aldosterone Enzyme Immunoassay kit (Cayman Chemical, Ann Arbor, Mich., USA). In brief, steroids were extracted from 60 μl of serum with ethyl acetate, dried and dissolved in 140 μl assay buffer, whereas 28 μl urine samples were diluted by five-folds with assay buffer. For measurement, 100 μl of serum extracts or urine diluents were incubated with 50 μl solutions containing DetectX aldosterone conjugate and antibody in coated 96-well plates overnight at 4° C. After washing with phosphate buffered saline (PBS) and adding the substrates for 30 minutes, the reactions were terminated and absorbance read at 450 nm using a plate reader (BioTEK Instrument Inc., Winooski, Vt., USA).

Antibody Production and Validation

Wild type human Lcn2 and the C87A mutant Lcn2 were expressed as His-tagged recombinant proteins and purified as previously described^{5, 6}. After removing endotoxin, the protein purity was confirmed by SDS-PAGE and mass spectrometry analysis. Polyclonal antibodies against human Lcn2 or C87A were produced as described previously^{5, 6}. The antibodies were purified by affinity chromatography for subsequent testing using human urine samples. Briefly, after removing the sediments by centrifugation at 2000 g for five minutes, 100 ml urine sample was concentrated to one ml using a Millipore® UFC900308 Amicon® Ultra-15 Centifugal Filter Concentrator with 3000 Da Nominal Molecular Weight Limit. Sixty μl of the concentrated urine samples were separated by SDS-PAGE, transferred to polyvinylidene difluoride membrane, and incubated with antibodies against either human wild-type Lcn2 or C87A mutant Lcn2. The immune-complexes were detected with chemiluminescence reagents from GE healthcare (Uppsala, Sweden). Both antibodies are able to detect Lcn2, however, with different patterns of migration and abundance (FIG. 4A).

For immunoprecipitation, the concentrated urine samples containing 500 μg proteins were diluted in 500 μl PBS, precleared with 50 μl protein A agarose beads slurry (Thermo Fisher Scientific, Waltham, Mass., USA), and then incubated overnight with 2.5 μg of antibodies against either human wild-type Lcn2 or C87A mutant Lcn2 at 4° C. under gentle agitation. Samples were then incubated with 100 μl of 50% protein A agarose beads slurry at room temperature for two hours under rotary agitation. After washing three times with PBS, the immune-complexes were eluted with SDS-PAGE loading buffer for Western blotting to detect the amount of polyamines attached to the precipitated Lcn2 using a specific antibody against spermidine or spermine. As the results shown in FIG. 4, the Lcn2 species precipitated by the antibody against C87A mutant Lcn2 contained barely detectable amount of polyamines when compared to those precipitated by the antibody against wild type human Lcn2 (FIG. 4B). Thus, the two antibodies selectively recognize npLcn2 and polyaminated Lcn2 (pLcn2), respectively.

Development of Sandwich ELISA

The sandwich ELISA for measuring human Lcn2 or polyaminated Lcn2 (pLcn2) was reported previously^{6, 21, 26, 27}. The sandwich ELISA for npLcn2 measurement was established by using unlabeled and biotinylated antibodies against C87A mutant Lcn2 for coating and detection, respectively. Biotinylation was performed with a kit from Thermo Scientific™ Pierce™ (Waltham, Mass., USA) and free biotin removed by dialysis. The microtiter plate was pre-coated with 100 μl unlabeled antibodies (2 μg/ml) overnight at 4° C., and then blocked with 100 μl of PBS containing 1% bovine serum albumin (BSA) and 0.05% Tween-20 for two hours at room temperature. For measurement, 100 μl diluted (25-fold) serum or non-diluted urine, or recombinant protein standards were applied into each well of the coated ELISA plates for one hour incubation at room temperature, followed by three times of washing and another hour of incubation with biotinylated antibodies. The bound immunocomplexes were detected with streptavidin-conjugated horseradish peroxidase and substrates. The reactions were stopped before measurement of the absorbance at 450 nm with a plate reader (Bio-TEK Instrument Inc.). The inter- and intra-assay coefficients of variance were determined by measuring six plasma samples from healthy subjects in a total of five independent assays with duplicate determinations.

Immunohistochemistry

Formalin-fixed, paraffin-embedded tissue sections (5 μm of thickness) were preheated at 60° C., deparaffinized, hydrated and then subjected for antigen retrieval in 0.01 M citrate buffer (pH 6.0). The endogenous peroxidase activity was quenched with 0.3% H₂O₂ for 15 minutes at room temperature. After blocking with 5% BSA in PBS for 30 minutes at room temperature, the tissue sections were incubated with anti-human Lcn2 antibody (5 μg/ml in PBS containing 5% BSA) or anti-C87A mutant Lcn2 antibody (5 μg/mL in PBS containing 5% BSA) overnight at 4° C. Anti-rabbit secondary antibody (1:1000, P0448, Dako, Denmark) was applied for 45 minutes at room temperature, followed by colorimetric detection with 3.3′-diaminobenzidine. All sections were counterstained with Mayer's hematoxylin prior to analysis under a microscope (BX51 Olympus, Japan) and with the Olympus cellSens Entry imaging software, version 1.7.

Targeted Disruption of Murine LCN2 Gene

The flippase recognition target (FRT)-Neo-FRT-loxP (1898 bp) was inserted in intron 1 and the loxP site located downstream of exon 2 of LCN2 (ENSMUSG00000026822, see world-wide website: ensembl.org/index.html). Following selection, positive ES clones were introduced into C57BL/6 blastocysts to produce chimeric mice, which were cross-bred with C57BL/6J mice. The Lcn2-floxed mice were obtained and subsequently crossed with the LysCre B6.129P2-Lyz2tm1(cre)Ifo/J mice from Jackson Laboratory (Bar Harbor, Me., USA), which expressed a Cre recombinase from the lysozyme M-encoding locus. The myeloid lineage-specific lipocalin-2 knockout mice (Lys-LKO) were produced and maintained in C57BL/6J background.

Neutrophil-Mesothelial Cell Interactions

The human mesothelial cell line MeT-5A (ATCC® CRL-9444™) was purchased from American Type Culture Collection (ATCC, Manassas, Va., USA), and cultured in M199 medium. After 48 hours, the conditioned media were collected for neutrophil cell incubation. Neutrophils were harvested from Lcn2 general knockout mice^{4, 5}. In brief, bone marrow cells were flushed out from femurs and tibias with Ca²⁺/Mg²⁺ free Hank's buffered saline solution supplemented with 20 mM Na-HEPES (pH 7.4). The cell suspension was filtered with a 70 micron cell strainer (Falcon #352350) and re-suspended for Percoll density gradient separations. After centrifuge at 1600 g for 30 min, neutrophils were collected between the layers of 78%, 69% and 52% Percoll and incubated with the conditioned medium collected from MeT-5A cultures for four hours at 37° C.

Statistical Analysis

All statistical calculations were performed with the IBM SPSS version 21.0 software (Chicago, Ill., USA). Data were expressed as mean±SD or median with interquartile range as appropriate. Kolmogorov-Smirnov test was used to analyze the distribution of different variables. Natural logarithmic transformation was applied for data with non-normal distribution. Independent T-test was used for comparison of continuous variables between two groups. Partial Pearson correlation was used to establish the relationship between variables of interest, with adjustment for age and gender. A p value less than 0.05 was used to indicate a significant difference in all statistical comparisons.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Gender Differences in LCN2, PLCN2, and NPLCN2 Levels

The levels of pLcn2 and npLcn2 in serum and urine samples collected from the community cohort were analyzed using in-house ELISA kits (Table 2). The average concentrations of pLcn2 in serum are comparable to those in urine samples, whereas the average concentrations of serum npLcn2 are higher by over 25-folds than those of urine npLcn2. Consequently, the ratios of pLcn2/npLcn2 are significantly lower for serum than urine samples.

TABLE 2
Lcn2 concentrations in samples collected from male
and female subjects of Hong Kong community cohort*.

| | Total cohort | Male | Female |
|---|---|---|---|
| | (mean ± SD) | (mean ± SD) | (mean ± SD) |
| Age, years | — | 48.2 ± 7.5 | 48.3 ± 7.2 |
| BMI, kg/m² | — | 24.7 ± 2.1^{a} | 22.7 ± 3.0 |
| WC, cm | — | 84.9 ± 5.4^{a} | 77.7 ± 8.0 |
| Serum pLcn2, ng/ml | 69.8 (42.3-126.4) | 85.9 (48.0-139.7)^{a} | 54.2 (33.7-79.1) |
| Serum npLcn2, ng/ml | 93.8 (63.1-143.3) | 102.6 (69.6-162.0)^{a} | 81.5 (54.2-105.1) |
| Serum pLcn2/npLcn2 | 0.81 (0.6-1.0) | 0.86 (0.7-1.1) | 0.76 (0.6-0.9) |
| Urine pLcn2, ng/ml | 60.8 (20.7-164.1) | 30.6 (16.3-78.0)^{a} | 161.0 (35.4-341.4) |
| Urine npLcn2, ng/ml | 3.7 (1.7-8.1) | 3.0 (1.6-5.0)^{a} | 6.5 (3.0-13.5) |
| Urine pLcn2/npLcn2 | 13.76 (10.7-21.4) | 13.03 (10.6-20.8) | 15.5 (11.8-22.6) |

^{a}P < 0.05 vs female subjects
*Data are shown as mean ± SD or median (interquartile range) values.

Compared to male subjects, both pLcn2 and npLcn2 were significantly decreased in serum samples of female participants; on the other hand, urine pLcn2 and npLcn2 levels in samples from female subjects were increased by five- and two-folds, respectively (Table 2). The gender differences of pLcn2 and npLcn2 in serum and urine samples remained after adjustment for BMI, WC and smoking. The ratios of pLcn2/npLcn2 are not significantly different between male and female subjects. There were no significant associations between serum or urine concentrations of Lcn2 and age, for both pLcn2 (p=0.303 and 0.875, respectively) and npLcn2 (p=0.315 and 0.555, respectively).

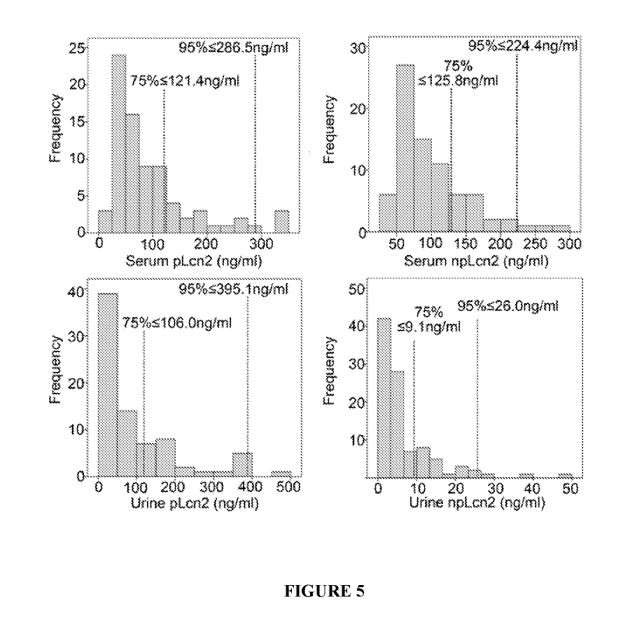

According to the guidelines of International Diabetes Federation for Chinese population²⁸, 31 subjects [14 men and 17 women, p=0.059] were centrally obese and had significantly higher pLcn2 (83.5 vs 46.1 ng/ml in non-obese subjects) and npLcn2 (5.2 vs 3.4 ng/ml in non-obese subjects) levels in urine (Table 3). In addition, serum npLcn2 levels were significantly higher in centrally obese female subjects. The 75^{th} percentile cut-off for serum and urinary Lcn2 levels in the healthy volunteers were 121.4 ng/ml and 106.0 ng/ml for pLcn2, and 125.8 ng/ml and 9.1 ng/ml for npLcn2, respectively (FIG. 5). The prevalence of metabolic syndrome were 10.2% in men and 19.5% in women (p=0.313). The 95^{th} percentile cut-off for serum and urinary Lcn2 levels in the heathy volunteers were determined to be 286.5 ng/ml and 395.1 ng/ml for pLcn2, and 224.4 ng/ml and 26.0 ng/ml for npLcn2, respectively (FIG. 4).

TABLE 3
Comparison of serum and urinary Lcn2 levels between non-obese
and centrally obese subjects in Hong Kong community cohort*.

| | Total cohort | | | Male | | | Female | | |
|---|---|---|---|---|---|---|---|---|---|
| | Non- | Centrally | | Non- | Centrally | | Non- | Centrally | |
| | obese | obese | | obese | obese | | obese | obese | |
| | (n = 69) | (n = 31) | p | (n = 45) | (n = 14) | p | (n = 24) | (n = 17) | p |
| Serum | 62.3 | 77.1 | 0.360 | 80.8 | 114.1 | 0.463 | 50.6 | 57.8 | 0.184 |
| plcn2 | (40.0-124.7) | (45.9-132.0) | | (45.5-146.6) | (65.7-204.9) | | (32.3-71.9) | (38.1-106.5) | |
| Serum | 86.9 | 95.8 | 0.693 | 98.9 | 118.5 | 0.595 | 65.0 | 94.6 | 0.04 |
| nplcn2 | (57.0-151.0) | (78.9-130.0) | | (68.8-163.3) | (76.5-158.4) | | (51.6-9.8) | (69.6-122.3) | |
| Urine | 46.1 | 83.5 | 0.031 | 31.3 | 25.0 | 0.519 | 98.1 | 205.2 | 0.011 |
| plcn2 | (18.6-111.5) | (21.0-330.0) | | (15.8-90.8) | (16.7-76.1) | | (23.6-187.9) | (66.1-413.0) | |
| Urine | 3.4 (1.6-5.9) | 5.2 | 0.044 | 2.8 | 2.7 | 0.567 | 5.2 | 11.9 | 0.048 |
| nplcn2 | | (2.5-12.4) | | (1.23-4.1) | (1.6-3.4) | | (1.8-12.5) | (5.5-16.2) | |

*Data are shown as median (interquartile range) values.

### EXAMPLE 2

### Correlation Analysis for Samples from Healthy Volunteers

In samples from community cohort, serum pLcn2 or npLcn2 levels are positively correlated with BMI, HR, DBP and TG, but negatively correlated with circulating concentrations of adiponectin, independent of age, gender and smoking (Table 4). After further adjustment for BMI, the positive correlations between npLcn2 and HR or TG remain significant. The ratios of serum pLcn2/npLcn2 are negatively correlated with TC (r=−0.299, p=0.004) and adiponectin (r=−0.253, p=0.015) levels, independent of age, gender, smoking and BMI.

TABLE 4
Correlations between serum Lcn2 concentrations and study variables.

| | serum plcn2^{#} | | | | serum nplcn2^{#} | | | |
|---|---|---|---|---|---|---|---|---|
| | r^{a} | r^{b} | p^{a} | p^{b} | r^{a} | r^{b} | p^{a} | p^{b} |
| BMI | 0.362 | — | 0.000 | — | 0.376 | — | 0.000 | — |
| WC | — | — | — | — | 0.236 | — | 0.020 | — |
| HR | 0.248 | — | 0.016 | — | 0.287 | 0.214 | 0.004 | 0.036 |
| SBP | — | — | — | — | — | | — | |
| DBP | 0.201 | — | 0.052 | — | 0.240 | | 0.018 | |
| FPG | — | — | — | — | — | — | — | — |
| TG | 0.289 | 0.211 | 0.005 | 0.042 | 0.354 | 0.283 | 0.000 | 0.005 |
| TC | — | — | — | — | — | — | — | — |
| HDL | — | — | — | — | — | — | — | — |
| LDL | — | — | — | — | — | — | — | — |
| Adiponectin^{#} | −0.330 | −0.222 | 0.001 | 0.034 | −0.297 | | 0.003 | — |

^{#}logarithmic transformed before analysis.
^{a}adjusted for age, gender and smoking.
^{b}adjusted for age, gender, smoking and BMI.

The concentrations of pLcn2 (p=0.960) or npLcn2 (p=0.312) in urine are not significantly correlated with those in serum samples. Smoking did not change Lcn2 levels in urine, but significantly increased the concentrations of serum pLcn2 and npLcn2 by 2.4- and 1.8-folds, respectively. After adjustment for age, gender and smoking, urinary npLcn2 levels are positively correlated with BMI, HR and serum TG concentrations (Table 5). The associations between pLcn2 or npLcn2 in urine and serum TG or urinary aldosterone levels remain significant after further adjustment for BMI.

TABLE 5
Correlations between urinary Lcn2
concentrations and study variables.

| | Urine plcn2 | | | | Urine nplcn2 | | | |
|---|---|---|---|---|---|---|---|---|
| | r^{a} | r^{b} | p^{a} | p^{b} | r^{a} | r^{b} | p^{a} | p^{b} |
| BMI | — | — | — | — | 0.214 | — | 0.018 | — |
| WC | 0.202 | 0.191 | 0.029 | 0.037 | — | — | — | — |
| HR | — | — | — | — | 0.167 | — | 0.051 | — |
| SBP | — | — | — | — | — | — | — | — |
| DBP | 0.175 | — | 0.050 | — | — | — | — | — |
| FPG | — | — | — | — | — | — | — | — |
| TG | 0.214 | 0.199 | 0.021 | 0.031 | 0.228 | 0.184 | 0.012 | 0.043 |
| TC | — | — | — | — | — | — | — | — |
| HDL | — | — | — | — | — | — | — | — |
| LDL | — | — | — | — | — | — | — | — |
| Serum | — | — | — | — | — | — | — | — |
| Aldosterone | | | | | | | | |
| Urine | 0.258 | 0.248 | 0.008 | 0.011 | 0.185 | 0.326 | 0.001 | 0.044 |
| Aldosterone ^{#} | | | | | | | | |

^{#} logarithmic transformed before analysis.
^{a}adjusted for age, gender, smoker
^{b}adjusted for age, gender, smoker and BMI

Urine samples contain a much higher level [1009.9 (482.5-2013.6) pg/ml] of aldosterone than that of serum samples [106.0 (77.7-131.3) pg/ml]. Aldosterone in urine is positively correlated with WC (r=0.275, p=0.023). Importantly, urinary pLcn2 (r=0.248, p=0.011) and npLcn2 (r=0.185, p=0.044) levels were positively correlated with the concentrations of aldosterone in urine, independent of age, gender, smoking and BMI (Table 5).

### EXAMPLE 3

### Correlation Analysis for Samples from Cardiothoracic Surgery Subjects

Among the 37 subjects in Danish cohort [31 male and 6 female subjects], most had been prescribed with various anti-coagulant, cholesterol lowering, renin angiotensin system inhibitory, anti-hypertensive and anti-diabetic medications¹⁶. When compared to those of the Hong Kong healthy volunteers, the ratios of plasma pLcn2/npLcn2 are reduced by over 4-folds, due mainly to the increased npLcn2 levels (Table 6). Both pLcn2 and npLcn2 are present in the pericardial fluids of all subjects, with a median ratio of 0.47 for pLcn2/npLcn2. The pLcn2 and npLcn2 levels in pericardial fluids are correlated significantly with those of the circulation.

TABLE 6
Concentrations between Lcn2 in plasma and pericardial fluid samples of Danish subjects.

| | | | | | | | Correlation between | |
|---|---|---|---|---|---|---|---|---|
| | Plasma | | | Pericardial fluid | | | plasma and | |
| | Total | | | Total | | | pericardial fluid | |
| | cohort | Male | Female | cohort | Male | Female | r | p |
| pLcn2 | 35.6 | 38.5 | 29.9 | 18.0 | 19.2 | 15.2 | 0.817 | 0.000 |
| | (28.4-46.7) | (29.9-51.9) | (24.7-63.0) | (14.0-26.7) | (12.9-28.7) | (14.0-35.5) | | |
| npLcn2 | 207.5 | 209.3 | 191.3 | 36.5 | 37.4 | 36.5 | 0.442 | 0.010 |
| | (162.8-249.7) | (164.9-254.2) | (130.7-272.7) | (28.9-51.5)^{c} | (28.8-61.4) | (34.0-69.8) | | |
| pLcn2/ | 0.19 | 0.20 | 0.20 | 0.47 | 0.47 | 0.43 | 0.028 | 0.876 |
| npLcn2 | (0.16-0.23) | (0.17-0.24) | (0.14-0.24) | (0.41-0.61) | (0.41-0.65) | (0.38-0.54) | | |

* Data are shown as median (interquartile range) values.

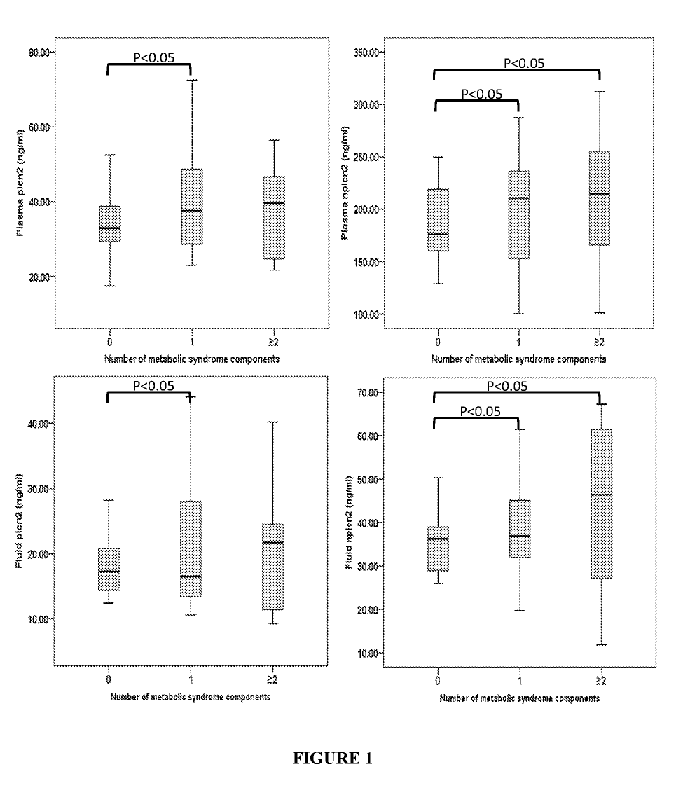

Based on National Cholesterol Education Program criteria²⁹, eight subjects of the Danish cohort with two or more components of metabolic syndrome had a significantly higher concentrations of nplcn2 levels in both circulation (241.9±54.2 ng/ml) and pericardial fluid (56.3±25.4 ng/ml) than the 15 subjects with none of the components (180.8±35.6 and 37.0±9.6 ng/ml for plasma and pericardial fluid, respectively). The nplcn2 levels in other subjects (n=14) were 213.0±51.6 and 49.5±21.6 ng/ml, respectively (FIG. 1). Positive associations are found between plasma levels of npLcn2 and CRP (r=0.359, p=0.017) or FGF21 (r=0.382, p=0.017). There were significant positive correlations of plasma creatinine concentrations with pLcn2 (r=0.404, p=0.023) or npLcn2 (r=0.630, p≤0.001) in pericardial fluids.

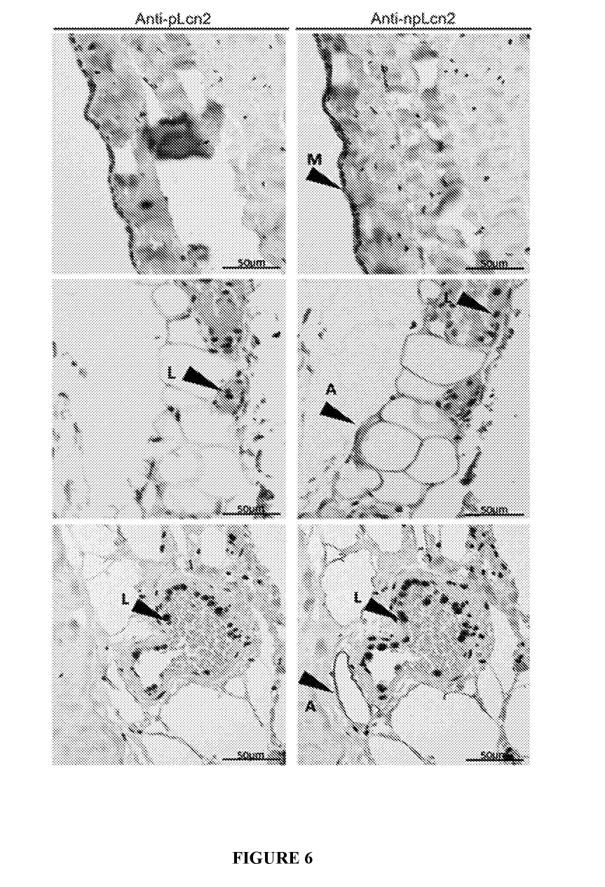

The presence and distribution of pLcn2 and npLcn2 were analyzed by immunohistochemical staining of the pericardial tissue biopsies (FIG. 6). While both pLcn2 and npLcn2 were detected in cells [referred to as leukocytes] located within or close to blood or lymph vessels, their patterns of distribution were significantly different when comparing the staining images from adjacent sections. The number of leukocytes containing pLcn2 was significantly less than those with npLcn2. Moreover, no positive staining for pLcn2 was found in cells of the paracardial adipose tissue and the mesothelial cell layer.

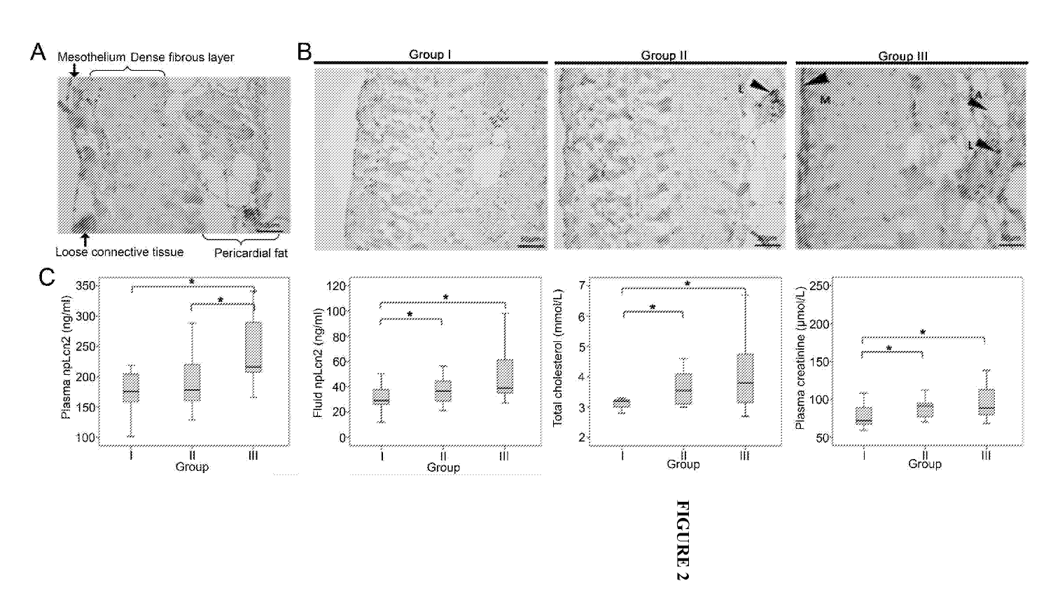

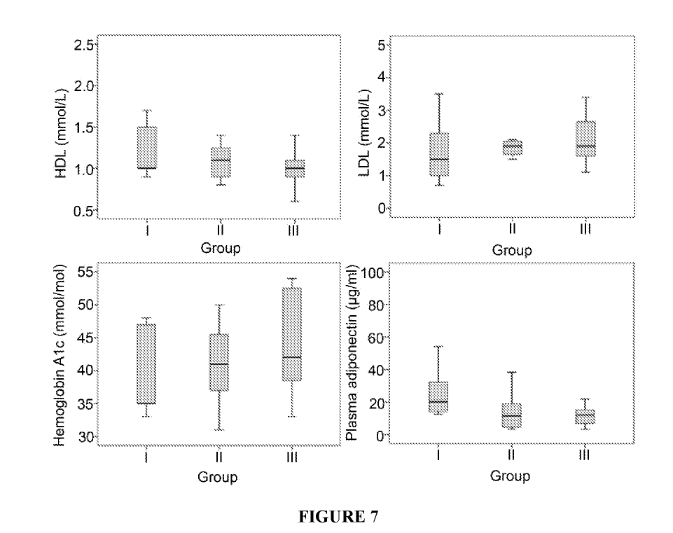

Based on the distribution of npLcn2 protein, samples were sorted into those with no or less than five positively stained cells (group I, n=9), or more than five positively stained cells [mainly leukocytes] (group II, n=11), and those with the positive staining widely distributed in leukocytes, adipocytes and mesothelial cells (group III, n=12). Compared to Group I and II, subjects in Group III exhibited the highest npLcn2 levels in both plasma and pericardial fluid samples. Compared to Group I, plasma TC and creatinine concentrations were significantly elevated in subjects from both Group II and III (FIG. 2). From group Ito group III, the circulating LDL levels and HbA1c) were progressively increasing, whereas the plasma HDL and adiponectin levels gradually decreased (FIG. 7). The pericardial fluid contents of CRP were the highest in samples from Group III (1710.7 ng/ml) subjects when compared to the other two groups (932.1 and 1149.8 ng/ml in Group I and II, respectively).

### REFERENCES

1. Flower D R. The lipocalin protein family: structure and function. The Biochemical journal. 1996; 318 (Pt 1):1-14.

2. Kjeldsen L, Johnsen A H, Sengelov H and Borregaard N. Isolation and primary structure of NGAL, a novel protein associated with human neutrophil gelatinase. J Biol Chem. 1993; 268:10425-32.

3. Flo T H, Smith K D, Sato S, Rodriguez D J, Holmes M A, Strong R K, Akira S and Aderem A. Lipocalin 2 mediates an innate immune response to bacterial infection by sequestrating iron. Nature. 2004; 432:917-21.

4. Law I K, Xu A, Lam K S, Berger T, Mak T W, Vanhoutte P M, Liu J T, Sweeney Zhou M, Yang B and Wang Y. Lcn2 deficiency attenuates insulin resistance associated with aging and obesity. Diabetes. 2010; 59:872-82.

5. Song E, Fan P, Huang B, Deng H B, Cheung BM, Feletou M, Vilaine J P, Villeneuve N, Xu A, Vanhoutte P M and Wang Y. Deamidated Lcn2 induces endothelial dysfunction and hypertension in dietary obese mice. Journal of the American Heart Association. 2014; 3:e000837.

6. Wang Y, Lam K S, Kraegen E W, Sweeney Zhang J, Tso A W, Chow W S, Wat N M, Xu J Y, Hoo R L and Xu A. Lcn2 is an inflammatory marker closely associated with obesity, insulin resistance, and hyperglycemia in humans. Clin Chem. 2007; 53:34-41.

7. Nickolas T L, Forster C S, Sise M E, Barasch N, Sola-Del Valle D, Viltard M, Buchen C, Kupferman S, Carnevali M L, Bennett M, Mattei S, Bovino A, Argentiero L, Magnano A, Devarajan P, Mori K, Erdjument-Bromage H, Tempst P, Allegri L and Barasch J. NGAL (Lcn2) monomer is associated with tubulointerstitial damage in chronic kidney disease. Kidney international. 2012; 82:718-22.

8. Tarjus A, Martinez-Martinez E, Amador C, Latouche C, El Moghrabi S, Berger T, Mak T W, Fay R, Farman N, Rossignol P, Zannad F, Lopez-Andres N and Jaisser F. Neutrophil Gelatinase-Associated Lipocalin, a Novel Mineralocorticoid Biotarget, Mediates Vascular Profibrotic Effects of Mineralocorticoids. Hypertension. 2015; 66:158-66.

9. Viau A, El Karoui K, Laouari D, Burtin M, Nguyen C, Mori K, Pillebout E, Berger T, Mak T W, Knebelmann B, Friedlander Barasch J and Terzi F. Lipocalin 2 is essential for chronic kidney disease progression in mice and humans. J Clin Invest. 2010; 120:4065-76.

10. Yndestad A, Landro L, Ueland T, Dahl C P, Flo T H, Vinge L E, Espevik T, Froland S S, Husberg C, Christensen Dickstein K, Kjekshus J, Oie E, Gullestad L and Aukrust P. Increased systemic and myocardial expression of neutrophil gelatinase-associated lipocalin in clinical and experimental heart failure. Eur Heart J. 2009; 30:1229-36.

11. Wang Y. Small lipid-binding proteins in regulating endothelial and vascular functions: focusing on adipocyte fatty acid binding protein and Lcn2. Br J Pharmacol. 2012; 165:603-21.

12. Liu J T, Song E, Xu A, Berger T, Mak T W, Tse H F, Law I K, Huang B, Liang Y, Vanhoutte P M and Wang Y. Lcn2 deficiency prevents endothelial dysfunction associated with dietary obesity: role of cytochrome P450 2C inhibition. Br J Pharmacol. 2012; 165:520-31.

13. Elneihoum A M, Falke P, Hedblad B, Lindgarde F and Ohlsson K. Leukocyte activation in atherosclerosis: correlation with risk factors. Atherosclerosis. 1997; 131:79-84.

14. Yan Q W, Yang Q, Mody N, Graham T E, Hsu C H, Xu Z, Houstis N E, Kahn B B and Rosen E D. The adipokine lipocalin 2 is regulated by obesity and promotes insulin resistance. Diabetes. 2007; 56:2533-40.

15. Leurgans T M, Bloksgaard M, Brewer J R, Bagatolli L A, Fredgart M H, Rosenstand K, Hansen M L, Rasmussen L M, Irmukhamedov A and De Mey J G. Endothelin-1 shifts the mediator of bradykinin-induced relaxation from NO to H2O2 in resistance arteries from subjects with cardiovascular disease. British journal of pharmacology. 2016; 173:1653-64.

16. Elie A G, Jensen P S, Nissen K D, Geraets I M, Xu A, Song E, Hansen M L, Irmukhamedov A, Rasmussen L M, Wang Y and De Mey J G. Adipokine Imbalance in the Pericardial Cavity of Cardiac and Vascular Disease Subjects. PloS one. 2016; 11:e0154693.

17. Xu A, Chan K W, Hoo R L, Wang Y, Tan K C, Zhang J, Chen B, Lam M C, Tse C, Cooper G J and Lam K S. Testosterone selectively reduces the high molecular weight form of adiponectin by inhibiting its secretion from adipocytes. J Biol Chem. 2005; 280:18073-80.

18. Lui M M, Lam J C, Mak H K, Xu A, Ooi C, Lam D C, Mak J C, Khong P L and Ip M S. C-reactive protein is associated with obstructive sleep apnea independent of visceral obesity. Chest. 2009; 135:950-6.

19. Yu H, Xia F, Lam K S, Wang Y, Bao Y, Zhang J, Gu Y, Zhou P, Lu J, Jia W and Xu A. Circadian rhythm of circulating fibroblast growth factor 21 is related to diurnal changes in fatty acids in humans. Clinical chemistry. 2011; 57:691-700.

20. Xu A, Wang Y, Xu J Y, Stejskal D, Tam S, Zhang J, Wat N M, Wong W K and Lam K S. Adipocyte fatty acid-binding protein is a plasma biomarker closely associated with obesity and metabolic syndrome. Clin Chem. 2006; 52:405-13.

21. Xiao Y, Xu A, Hui X, Zhou P, Li X, Zhong H, Tang W, Huang G and Zhou Z. Circulating Lcn2 and retinol-binding protein 4 are associated with intima-media thickness and subclinical atherosclerosis in subjects with type 2 diabetes. PloS one. 2013; 8:e66607.

22. Lin Z, Tian H, Lam K S, Lin S, Hoo R C, Konishi M, Itoh N, Wang Y, Bornstein S R, Xu A and Li X. Adiponectin mediates the metabolic effects of FGF21 on glucose homeostasis and insulin sensitivity in mice. Cell metabolism. 2013; 17:779-89.

23. Xiao Y, Xu A, Law L S, Chen C, Li H, Li X, Yang L, Liu S, Zhou Z and Lam K S. Distinct changes in serum fibroblast growth factor 21 levels in different subtypes of diabetes. The Journal of clinical endocrinology and metabolism. 2012; 97:E54-8.

24. Lin Z, Gong Q, Wu C, Yu J, Lu T, Pan X, Lin S and Li X. Dynamic change of serum FGF21 levels in response to glucose challenge in human. The Journal of clinical endocrinology and metabolism. 2012; 97:E1224-8.

25. Zhou M, Bao Y, Lu J, Zhou J and Jia W. Serum A-FABP is increased and closely associated with elevated NT-proBNP levels in type 2 diabetic subjects treated with rosiglitazone. PloS one. 2011; 6:e27032.

26. Milner K L, van der Poorten D, Xu A, Bugianesi E, Kench J G, Lam K S, Chisholm D J and George J. Adipocyte fatty acid binding protein levels relate to inflammation and fibrosis in nonalcoholic fatty liver disease. Hepatology. 2009; 49:1926-34.

27. Alkharfy K M, Al-Daghri N M, Vanhoutte P M, Krishnaswamy S and Xu A. Serum retinol-binding protein 4 as a marker for cardiovascular disease in women. PloS one. 2012; 7:e48612.

28. Alberti K G, Zimmet P and Shaw J. Metabolic syndrome—a new world-wide definition. A Consensus Statement from the International Diabetes Federation. Diabetic medicine: a journal of the British Diabetic Association. 2006; 23:469-80.

29. Third Report of the National Cholesterol Education Program (NCEP) Expert Panel on Detection, Evaluation, and Treatment of High Blood Cholesterol in Adults (Adult Treatment Panel III) final report. Circulation. 2002; 106:3143-421.

30. Zhang Y, Foncea R, Deis J A, Guo H, Bernlohr D A and Chen X. Lipocalin 2 expression and secretion is highly regulated by metabolic stress, cytokines, and nutrients in adipocytes. PloS one. 2014; 9:e96997.

31. Nilsen-Hamilton M, Hamilton R T and Adams G A. Rapid selective stimulation by growth factors of the incorporation by BALB/C 3T3 cells of [35S]methionine into a glycoprotein and five superinducible proteins. Biochemical and biophysical research communications. 1982; 108:158-66.

32. Pawluczyk I Z, Furness P N and Harris K P. Macrophage-induced rat mesangial cell expression of the 24p3-like protein alpha-2-microglobulin-related protein. Biochim Biophys Acta. 2003; 1645:218-27.

33. Meheus L A, Fransen L M, Raymackers J G, Blockx H A, Van Beeumen J J, Van Bun S M and Van de Voorde A. Identification by microsequencing of lipopolysaccharide-induced proteins secreted by mouse macrophages. J Immunol. 1993; 151:1535-47.

34. Challen G A, Martinez Davis M J, Taylor D F, Crowe M, Teasdale R D, Grimmond S M and Little M R. Identifying the molecular phenotype of renal progenitor cells. Journal of the American Society of Nephrology: JASN. 2004; 15:2344-57.

35. Hraba-Renevey S, Turler H, Kress M, Salomon C and Weil R. SV40-induced expression of mouse gene 24p3 involves a post-transcriptional mechanism. Oncogene. 1989; 4:601-8.

36. Nielsen B S, Borregaard N, Bundgaard J R, Timshel S, Sehested M and Kjeldsen L. Induction of NGAL synthesis in epithelial cells of human colorectal neoplasia and inflammatory bowel diseases. Gut. 1996; 38:414-20.

37. Stoesz S P and Gould M N. Overexpression of neu-related lipocalin (NRL) in neu-initiated but not ras or chemically initiated rat mammary carcinomas. Oncogene. 1995; 11:2233-41.

38. Clemmensen S N, Bohr C T, Rorvig S, Glenthoj A, Mora-Jensen H, Cramer E P, Jacobsen L C, Larsen M T, Cowland J B, Tanassi J T, Heegaard N H, Wren J D, Silahtaroglu A N and Borregaard N. Olfactomedin 4 defines a subset of human neutrophils. Journal of leukocyte biology. 2012; 91:495-500.

39. Le Cabec V, Cowland J B, Calafat J and Borregaard N. Targeting of proteins to granule subsets is determined by timing and not by sorting: The specific granule protein NGAL is localized to azurophil granules when expressed in HL-60 cells. Proceedings of the National Academy of Sciences of the United States of America. 1996; 93:6454-7.

40. Borregaard N, Sehested M, Nielsen B S, Sengelov H and Kjeldsen L. Biosynthesis of granule proteins in normal human bone marrow cells. Gelatinase is a marker of terminal neutrophil differentiation. Blood. 1995; 85:812-7.

41. Cowland J B and Borregaard N. Molecular characterization and pattern of tissue expression of the gene for neutrophil gelatinase-associated lipocalin from humans. Genomics. 1997; 45:17-23.

42. Auguet T, Terra X, Quintero Y, Martinez S, Manresa N, Porras J A, Aguilar C, Orellana-Gavalda J M, Hernandez M, Sabench F, Lucas A, Pellitero S, Del Castillo D and Richart C. Liver lipocalin 2 expression in severely obese women with non alcoholic fatty liver disease. Experimental and clinical endocrinology & diabetes: official journal, German Society of Endocrinology [and] German Diabetes Association. 2013; 121:119-24.

43. Gineste A, Martin P, Oswald E, Coppin H and Roth M P. Evidence for IL-6/STAT3-independent induction of Lcn2 in the liver of mice infected with Escherichia coli. Hepatology (Baltimore, Md.). 2016; 63:673-4.

44. Garay-Rojas E, Harper M, Hraba-Renevey S and Kress M. An apparent autocrine mechanism amplifies the dexamethasone- and retinoic acid-induced expression of mouse Lcnencoding gene 24p3. Gene. 1996; 170:173-80.

45. Le Cabec V, Calafat J and Borregaard N. Sorting of the specific granule protein, NGAL, during granulocytic maturation of HL-60 cells. Blood. 1997; 89:2113-21.

46. Hochmeister S, Engel O, Adzemovic M Z, Pekar T, Kendlbacher P, Zeitelhofer M, Haindl M, Meisel A, Fazekas F and Seifert-Held T. Lcn2 as an Infection-Related Biomarker to Predict Clinical Outcome in Ischemic Stroke. PloS one. 2016; 11:e0154797.

47. Ye D, Yang K, Zang S, Lin Z, Chau H T, Wang Y, Zhang J, Shi J, Xu A and Lin S. Lcn2 Mediates Nonalcoholic Steatohepatitis by Promoting Neutrophil-Macrophage Crosstalk via The Induction of CXCR2. Journal of hepatology. 2016.

48. Fjaertoft Foucard T, Xu S and Venge P. Human neutrophil lipocalin (HNL) as a diagnostic tool in children with acute infections: a study of the kinetics. Acta Paediatr. 2005; 94:661-6.

49. Liu Q and Nilsen-Hamilton M. Identification of a new acute phase protein. J Biol Chem. 1995; 270:22565-70.

50. Chan Y K, Sung H K, Jahng J W, Kim G H, Han M and Sweeney G. Lcn2 inhibits autophagy and induces insulin resistance in H9c2 cells. Molecular and cellular endocrinology. 2016; 430:68-76.

51. Kjeldsen L, Bainton D F, Sengelov H and Borregaard N. Identification of neutrophil gelatinase-associated lipocalin as a novel matrix protein of specific granules in human neutrophils. Blood. 1994; 83:799-807.

52. Axelsson L, Bergenfeldt M and Ohlsson K. Studies of the release and turnover of a human neutrophil lipocalin. Scand J Clin Lab Invest. 1995; 55:577-88.

53. Mori K, Lee H T, Rapoport D, Drexler I R, Foster K, Yang J, Schmidt-Ott K M, Chen X, Li J Y, Weiss S, Mishra J, Cheema F H, Markowitz Suganami T, Sawai K, Mukoyama M, Kunis C, D'Agati V, Devarajan P and Barasch J. Endocytic delivery of Lcnsiderophore-iron complex rescues the kidney from ischemia-reperfusion injury. J Clin Invest. 2005; 115:610-21.

54. Donadio C. Effect of glomerular filtration rate impairment on diagnostic performance of neutrophil gelatinase-associated lipocalin and B-type natriuretic peptide as markers of acute cardiac and renal failure in chronic kidney disease subjects. Critical care (London, England). 2014; 18:R39.

55. Clerico A, Galli C, Fortunato A and Ronco C. Neutrophil gelatinase-associated lipocalin (NGAL) as biomarker of acute kidney injury: a review of the laboratory characteristics and clinical evidences. Clinical chemistry and laboratory medicine. 2012; 50:1505-17.

56. Cullen M R, Murray P T and Fitzgibbon M C. Establishment of a reference interval for urinary neutrophil gelatinase-associated lipocalin. Annals of clinical biochemistry. 2012; 49:190-3.

57. Dieterle F, Perentes E, Cordier A, Roth D R, Verdes P, Grenet O, Pantano S, Moulin P, Wahl D, Mahl A, End P, Staedtler F, Legay F, Carl K, Laurie D, Chibout S D, Vonderscher J and Maurer G Urinary clusterin, cystatin C, beta2-microglobulin and total protein as markers to detect drug-induced kidney injury. Nature biotechnology. 2010; 28:463-9.

58. Hoffmann D, Fuchs T C, Henzler T, Matheis K A, Herget T, Dekant W, Hewitt P and Mally A. Evaluation of a urinary kidney biomarker panel in rat models of acute and subchronic nephrotoxicity. Toxicology. 2010; 277:49-58.

59. Wagener Jan M, Kim M, Mori K, Barasch J M, Sladen R N and Lee H T. Association between increases in urinary neutrophil gelatinase-associated lipocalin and acute renal dysfunction after adult cardiac surgery. Anesthesiology. 2006; 105:485-91.

60. Barrera-Chimal J and Bobadilla N A. Are recently reported biomarkers helpful for early and accurate diagnosis of acute kidney injury? Biomarkers: biochemical indicators of exposure, response, and susceptibility to chemicals. 2012; 17:385-93.

61. Waikar S S and Bonventre J V. Biomarkers for the diagnosis of acute kidney injury. Nephron Clinical practice. 2008; 109:c192-7.

62. Schmidt-Ott K M, Mori K, Kalandadze A, Li J Y, Paragas N, Nicholas T, Devarajan P and Barasch J. Neutrophil gelatinase-associated Lcnmediated iron traffic in kidney epithelia. Curr Opin Nephrol Hypertens. 2006; 15:442-9.

63. Schmidt-Ott K M, Mori K, Li J Y, Kalandadze A, Cohen D J, Devarajan P and Barasch J. Dual action of neutrophil gelatinase-associated lipocalin. Journal of the American Society of Nephrology: JASN. 2007; 18:407-13.

64. Palazzuoli A, Ruocco Beltrami M, Franci B, Pellegrini M, Lucani B, Nuti R and Ronco C. Admission plasma neutrophil gelatinase associated lipocalin (NGAL) predicts worsening renal function during hospitalization and post discharge outcome in subjects with acute heart failure. Acute cardiac care. 2014; 16:93-101.

65. Mori K and Nakao K. Neutrophil gelatinase-associated lipocalin as the real-time indicator of active kidney damage. Kidney international. 2007; 71:967-70.

66. Haase M, Bellomo R, Devarajan P, Schlattmann P and Haase-Fielitz A. Accuracy of neutrophil gelatinase-associated lipocalin (NGAL) in diagnosis and prognosis in acute kidney injury: a systematic review and meta-analysis. Am J Kidney Dis. 2009; 54:1012-24.

67. Alvelos M, Pimentel R, Pinho E, Gomes A, Lourenco P, Teles M J, Almeida P, Guimaraes J T and Bettencourt P. Neutrophil gelatinase-associated lipocalin in the diagnosis of type 1 cardio-renal syndrome in the general ward. Clinical journal of the American Society of Nephrology: CJASN. 2011; 6:476-81.

68. Iozzo P. Myocardial, perivascular, and epicardial fat. Diabetes care. 2011; 34 Suppl 2:S371-9.

69. Chau Y Y, Bandiera R, Serrels A, Martinez-Estrada O M, Qing W, Lee M, Slight J, Thornburn A, Berry R, McHaffie S, Stimson R H, Walker B R, Chapuli R M, Schedl A and Hastie N. Visceral and subcutaneous fat have different origins and evidence supports a mesothelial source. Nature cell biology. 2014; 16:367-75.

70. Vogiatzidis K, Zarogiannis S G, Aidonidis I, Solenov E I, Molyvdas P A, Gourgoulianis K I and Hatzoglou C. Physiology of pericardial fluid production and drainage. Frontiers in physiology. 2015; 6:62.

71. Jobsis P D, Ashikaga H, Wen H, Rothstein E C, Horvath K A, McVeigh E R and Balaban R S. The visceral pericardium: macromolecular structure and contribution to passive mechanical properties of the left ventricle. American journal of physiology Heart and circulatory physiology. 2007; 293:H3379-87.

72. Benhaiem-Sigaux N, Mina E, Sigaux F, Lambre C R, Valensi F, Allegret C and Bernaudin J F. Characterization of human pericardial macrophages. Journal of leukocyte biology. 1985; 38:709-21.

73. Harvey D J, Proteomic analysis of glycosylation: structural determination of N- and O-linked glycans by mass spectrometry. Expert Review of Proteomics 2005, 2(1):87-101.

74. Greenfield, E., (2014), Antibodies: A Laboratory Manual, Second Edition, Cold Spring Harbor, N.Y.: Cold Spring Harbor Laboratory Press.

1. A method for treating and/or managing and/or preventing a cardiometabolic disease in a subject, the method comprising the steps of:
(a) determining the level of non-polyaminated lipocalin-2 (npLcn2) in:
i) a test sample obtained from the subject, and
ii) optionally a control sample;
(b) optionally, obtaining one or more reference values corresponding to the level of npLcn2; and
(c) identifying the subject as:
i) having or having high risk of developing the cardiometabolic disease based on the level of npLcn2 in the test sample and optionally, administering to the subject a therapy designed to treat and/or manage and/or prevent the cardiometabolic disease, or
ii) identifying the subject as not having or having low risk of developing the cardiometabolic disease based on the level of npLcn2 in the test sample and withholding from the subject the therapy designed to treat and/or manage and/or prevent the cardiometabolic disease.

2. The method of claim 1, wherein the therapy to treat and/or manage and/or prevent the cardiometabolic disease comprises administering to the subject an antibody that specifically binds to pLcn2 or npLcn2.

3. The method of claim 1, wherein the control sample is obtained from one or more of the following: an individual belonging to the same species as the subject and not having the cardiometabolic disease, an individual belonging to the same species as the subject and known to have a low risk or no risk of developing the cardiometabolic disease; the subject prior to having the cardiometabolic disease.

4. The method of claim 2, wherein the control sample and the test sample are obtained from the same type of an organ or tissue.

5. The method of claim 3, wherein the organ or tissue is placenta, brain, eye, pineal gland, pituitary gland, thyroid gland, parathyroid gland, thorax, heart, lung, esophagus, thymus gland, pleura, adrenal gland, appendix, gall bladder, urinary bladder, large intestine, small intestine, kidney, liver, pancreas, spleen, stoma, ovary, uterus, skin, blood, or buffy coat sample of blood.

6. The method of claim 2, wherein the control sample and the test sample are obtained from the same type of a body fluid.

7. The method of claim 5, wherein the body fluid is amniotic fluid, aqueous humor, vitreous humor, bile, blood, cerebrospinal fluid, chyle, endolymph, perilymph, female ejaculate, lymph, mucus (including nasal drainage and phlegm), pericardial fluid, peritoneal fluid, pleural fluid, pus, rheum, saliva, sputum, synovial fluid, vaginal secretion, blood, serum, or plasma.

8. The method of claim 1, wherein the level of pLcn2 and/or npLcn2 in the test sample and optionally, the control sample, are determined by an antibody based assay or protein mass-spectrometry.

9. The method of claim 8, wherein the antibody based assay is western blot analysis, enzyme immunoassay (EIA), enzyme linked immunosorbent assay (ELISA), radioimmune assay (RIA), immunehistological analysis, or antigen-antibody precipitation assay.

10. The method of claim 1, wherein the subject is a mammal.

11. The method of claim 10, wherein the mammal is a human, ape, canine, pig, bovine, rodent, or feline.

12. An antibody or an antigen binding fragment of an antibody that specifically binds to pLcn2 or npLcn2.

13. The antibody or the antigen fragment thereof of claim 12, selected from chimeric antibody, humanized monoclonal antibody, a single chain antibody, a single chain fragment variable (scFv) antibody, and a fragment antigen-binding (Fab fragment).

14. An assay comprising determining the level npLcn2 in:
i) a test sample obtained from the subject, and
ii) optionally, a control sample.

15. The assay of claim 14, wherein the assay is an antibody based assay or protein mass-spectrometry.

16. The assay of claim 15, wherein the antibody based assay is western blot analysis, EIA, ELISA, RIA, immunehistological analysis, or antigen-antibody precipitation assay.

17. An assay for determining whether the level of pLcn2 and/or npLcn2 in a test sample obtained from a subject is above or below a threshold level, the assay comprising the steps of:
a) determining the level of pLcn2 and/or npLcn2 in:
i) a test sample obtained from the subject, and
ii) optionally, a control sample; and
b) determining whether the level of pLcn2 and/or npLcn2 in a test sample obtained from a subject is above or below the threshold level.

18. The method of claim 17, wherein the test sample is a urine sample and the threshold level for pLcn2 is about 110 ng/ml or about 400 ng/ml.

19. The method of claim 17, wherein the test sample is a urine sample and the threshold level for npLcn2 is about 11 ng/ml or 26 ng/ml.

20. A device that indicates whether the level of pLcn2 and/or npLcn2 in a test sample obtained from a subject is above or below a threshold level, wherein the device provides a first signal if the level of pLcn2 and/or npLcn2 in a test sample obtained from a subject is above a threshold level and the device provides a second signal if the level of pLcn2 and/or npLcn2 in the test sample obtained from the subject is below the threshold level.

21. A device that indicates whether the level of pLcn2 and/or npLcn2 in a test sample obtained from a subject is above or below a threshold level by providing a signal, wherein the device provides the signal only if the level of pLcn2 and/or npLcn2 in a test sample obtained from a subject is above the threshold level or the device provides the signal only if the level of pLcn2 and/or npLcn2 in the test sample obtained from the subject is below the threshold level.
